(19) United States
(12) Patent Application Publication
(10) Pub. No.: US 2022/0331145 A1
(43) Pub. Date: Oct. 20, 2022

(54) URINE COLLECTION APPARATUS

(71) Applicant: AMI Industries, Inc., Colorado Springs, CO (US)

(72) Inventors: Kayla Goodrich, Colorado Springs, CO (US); Amanda Mattox, Fountain, CO (US); Tristan Likes, Colorado Springs, CO (US); Claire Callahan, Marion, IA (US)

(73) Assignee: AMI Industries, Inc., Colorado Springs, CO (US)

(21) Appl. No.: 17/233,334

(22) Filed: Apr. 16, 2021

(51) Int. Cl. A61F 5/455; A61F 5/44

(52) U.S. Cl. CPC A61F 5/455; A61F 5/4404

(57) ABSTRACT

A urine collection apparatus for human females may comprise an interface body including a cup portion and an elongate portion, the cup portion and the elongate portion defining an interface surface engaging the user's body, wherein the cup portion is forward of the elongate portion and sized to be disposed in surrounding relation to the urethral opening of the user while the user is in a seated position, and wherein the elongate portion is configured to be disposed under the user while the user is in a seated position.

### FIELD

The present disclosure generally relates to urine collection devices and, more particularly, for such devices for use by females.

### BACKGROUND

Certain occupations may preclude access to ordinary restroom facilities for extended periods of time. For example, a pilot may be strapped into their seat and/or various safety apparatuses. Traditional urine collection apparatuses may require the pilot to loosen and/or remove the various harness straps to reposition their body and, in this regard, may tend to compromise pilot safety.

### SUMMARY

In various embodiments, a urine collection apparatus is disclosed. The urine collection apparatus may comprise an interface body including a cup portion and an elongate portion, the cup portion and the elongate portion defining an interface surface engaging the user's body, wherein the cup portion is forward of the elongate portion and sized to be disposed in surrounding relation to the urethral opening of the user while the user is in a seated position, and wherein the elongate portion is configured to slide under the user while the user is in a seated position.

In various embodiments, the cup portion comprises a material which is resilient and flexible at human body temperature and pressure such that when in contact with the temperature and pressure of the human body in use, it will soften to conform with the configuration of the anatomy of the user. In various embodiments, the cup portion comprises a funnel portion. In various embodiments, the funnel portion comprises a coupling portion at the base of the funnel portion. In various embodiments, the coupling portion comprises a threaded coupling. In various embodiments, the interface body comprises a polymeric or elastomeric material. In various embodiments, the cup portion and the elongate portion are overmolded over the interface body.

In various embodiments, a urine collection system is disclosed comprising a urine collection reservoir, a urine collection apparatus for human females, and a collection tube configured to couple to the urine collection reservoir and a urine collection apparatus and enable fluid communication therebetween, wherein the urine collection apparatus for human females comprises an interface body including a cup portion and an elongate portion, the cup portion and the elongate portion defining an interface surface engaging the user's body, wherein the cup portion is forward of the elongate portion and sized to be disposed in surrounding relation to the urethral opening of the user while the user is in a seated position, and wherein the elongate portion is configured to slide under the user while the user is in a seated position.

In various embodiments, the collection tube comprises a plurality of bellows segments. In various embodiments, the urine collection reservoir comprises a flexible containment bag. In various embodiments, the cup portion comprises a funnel portion, wherein the funnel portion comprises a first coupling portion at the base of the funnel portion. In various embodiments, the collection tube comprises a second coupling portion and a third coupling portion, wherein the plurality of bellows segments are disposed between the second coupling portion and the third coupling portion. In various embodiments, the urine collection reservoir comprises a fourth coupling portion, wherein each of the first coupling portion, the second coupling portion, the third coupling portion, and the fourth coupling portion comprise threaded couplings.

In various embodiments a method of installing a urine collection system comprises sliding an elongate portion of an interface body under a user while the user is in the seated position, disposing a cup portion of the interface body in surrounding relation to a urethral opening of the user in response to sliding the elongate portion under the user, transitioning a collection tube from a stowed condition to an expanded condition, coupling a first coupling portion of a funnel portion of the cup portion to a second coupling portion of the collection tube, and coupling a third coupling portion of the collection tube to a fourth coupling portion of a urine collection reservoir.

The foregoing features and elements may be combined in various combinations without exclusivity, unless expressly indicated herein otherwise. These features and elements as well as the operation of the disclosed embodiments will become more apparent in light of the following description and accompanying drawings.

### BRIEF DESCRIPTION OF THE DRAWINGS

The subject matter of the present disclosure is particularly pointed out and distinctly claimed in the concluding portion of the specification. A more complete understanding of the present disclosure, however, may be obtained by referring to the detailed description and claims when considered in connection with the drawing figures, wherein like numerals denote like elements.

FIG. 1 is ventral-dorsal cross-sectional view through a human body and a urine collection device, according to various embodiments;

FIG. 2 illustrates a system for a urine collection, in accordance with various embodiments;

FIG. 3A illustrates a collection tube in a system for a urine collection device in a stowed condition;

FIG. 3B illustrates a collection tube in a system for urine collection in an expanded condition, in accordance with various embodiments; and

FIG. 4 illustrates a collection reservoir, in accordance with various embodiments.

### DETAILED DESCRIPTION

The detailed description of exemplary embodiments herein makes reference to the accompanying drawings, which show exemplary embodiments by way of illustration and their best mode. While these exemplary embodiments are described in sufficient detail to enable those skilled in the art to practice the disclosures, it should be understood that other embodiments may be realized and that logical, chemical, and mechanical changes may be made without departing from the spirit and scope of the disclosures. Thus, the detailed description herein is presented for purposes of illustration only and not of limitation. For example, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

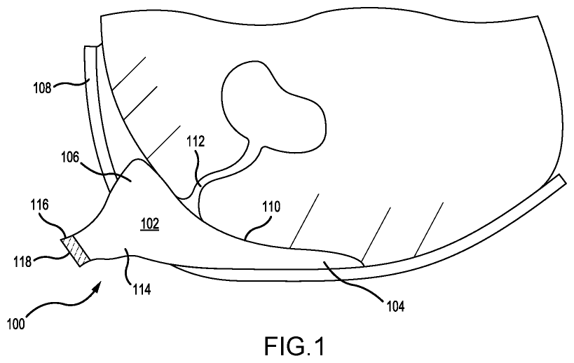

In various embodiments, and with reference to FIG. 1, a urine collection apparatus is 100 shown interfaced with a user who is occupationally prohibited from using ordinary restroom facilities for extended periods of time, for example, by a pilot of an aircraft (e.g. a light aircraft). The device includes an interface body 102 configured to interface with the anatomy of the user while the user is in a sitting position. The interface body 102 includes a cup portion 106 and an elongate portion 104 which collectively define an interface surface 110 with the user's anatomy. In various embodiments, the interface body 102 may comprise a polymeric or elastomeric material such as, for example, one of a silicone, a rubber, an over molded plastic, and/or the like. To enhance comfort, the cup portion 106 and/or elongate portion 104 may comprise a material or combination of materials which is resilient and flexible at human body temperature and pressure such that when in contact with the temperature and pressure of the human body in use, it will soften somewhat and tend to conform even more perfectly with the configuration of the anatomy of the individual user.

The cup portion 106 is sized and positioned to be disposed in surrounding relation to the urethral opening 112 of the user while the user is in a seated position. In this regard, the cup portion 106 is forward of the elongate portion 104. The elongate portion 104 extends from a lower edge of the cup portion 106. In various embodiments, the cup portion 106 and/or the elongate portion 104 may be overmolded over the interface body 102. The elongate portion 104 is configured to be disposed under the user when in a seated position and thereby assist in retaining the cup portion 106 proximate the urethral opening 112 of the user. In various embodiments, the elongate portion 104 may be shaped to slide between the user and a support surface (such as, for example, an aircraft seat) when the user is in a seated position on the support surface and without the user rising from the seated position or otherwise breaking contact with the support surface. In various embodiments, the cup portion 106 may be further retained against the user by a garment 108 such as, for example, a flight suit or undergarment through which the cup portion 106 and the elongate portion 104 may be inserted. In this regard, the garment 108 may tend to retain the interface body 102 proximate the user's anatomy. In various embodiments, the cup portion 106 includes a funnel portion 114. The funnel portion 114 is configured to collect the urine from the cup portion 106 and direct the urine toward a first coupling portion 116 at the base of the funnel portion 114. In various embodiments, the first coupling portion 116 comprises an opening 118 which is in fluid communication with the funnel portion 114. A removable cap may be fitted to the opening and/or may be coupled to the first coupling portion 116 and thereby the opening 118 may be sealed to inhibit fluid communication. In various embodiments, the first coupling portion 116 may comprise a threaded coupling. In various embodiments, the cap may be threaded to the threaded coupling.

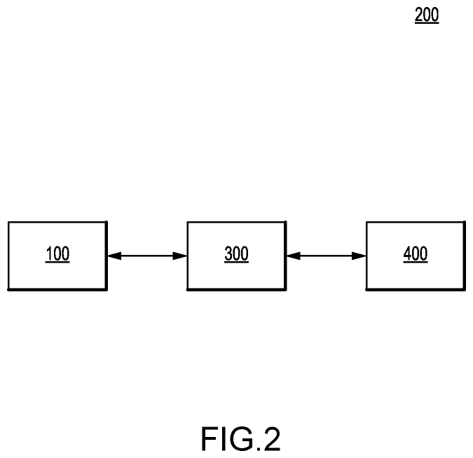

In various embodiments and with additional reference to FIG. 2 a system 200 for urine collection is illustrated. System 200 includes urine collection apparatus 100, a urine collection reservoir 400 (i.e. reservoir 400), and a collection tube 300 configured to couple with the reservoir 400 and the first coupling portion 116 of the urine collection apparatus 100. System 200 enables fluid communication between the urine collection apparatus 100 and the reservoir 400 and thereby allows urine to be escorted away from the user for storage and later disposal.

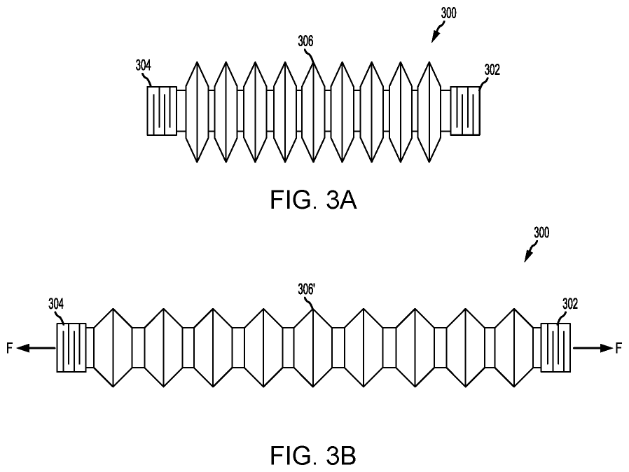

With additional reference to FIGS. 3A and 3B, collection tube 300 is illustrated in accordance with various embodiments. FIG. 3A shows collection tube 300 in the stowed condition and FIG. 3B shows collection tube 300 in the expanded condition. Collection tube 300 includes a second coupling portion 302 and a third coupling portion 304. In various embodiments, the second coupling portion 302 may be configured to couple with the first coupling portion 116 and the third coupling portion 304 may be configured to couple to the reservoir 400. In various embodiments, the coupling portions (302, 304) may comprise threaded couplings. Fluid communication is enabled between the coupling portions (302, 304) via a plurality of expandable bellows segments such as, for example, bellows segment 306 which are disposed between the coupling portions (302, 304). In this regard, the second coupling portion 302 is distal of the third coupling portion 304. As shown in FIG. 3B, applying a force F at the coupling portions (302, 304) may tend to expand the plurality of bellows segments 306 to an expanded state (e.g., expanded bellows segment 306′) and thereby increase the length of the collection tube 300. In like regard, application of an opposite force (or reduction/removal of the force F) may tend to cause the plurality of bellows segments to transition from the expanded condition back toward the stowed condition.

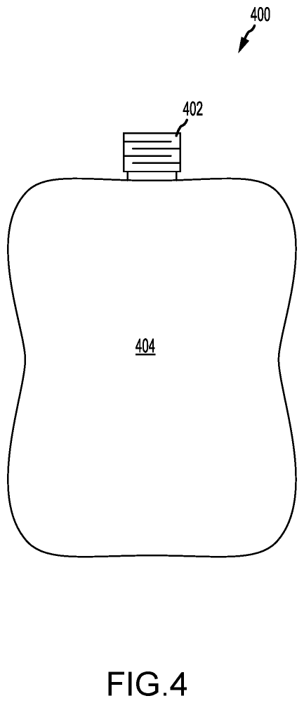

With additional reference to FIG. 4, reservoir 400 is illustrated in accordance with various embodiments. Reservoir 400 comprises a fourth coupling portion 402 in fluid communication with a flexible containment bag 404. In various embodiments, containment bag 404 comprises a flexible material impermeable to liquid. In this regard, containment bag 404 may be collapsed or rolled up for storage.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosures.

The scope of the disclosures is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods and apparatus are provided herein. In the detailed description herein, references to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiment.

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A urine collection apparatus for human females, comprising:
an interface body including a cup portion and an elongate portion, the cup portion and the elongate portion defining an interface surface engaging the user's body,
wherein the cup portion is forward of the elongate portion and sized to be disposed in surrounding relation to the urethral opening of the user while the user is in a seated position, and
wherein the elongate portion is configured to be disposed under the user while the user is in a seated position.

2. The urine collection apparatus of claim 1, wherein the cup portion comprises a material which is resilient and flexible at human body temperature and pressure such that when in contact with the temperature and pressure of the human body in use, it will soften to conform with the configuration of the anatomy of the user.

3. The urine collection apparatus of claim 1, wherein the cup portion comprises a funnel portion.

4. The urine collection apparatus of claim 3, wherein the funnel portion comprises a coupling portion at a base of the funnel portion.

5. The urine collection apparatus of claim 4, wherein the coupling portion comprises a threaded coupling.

6. The urine collection apparatus of claim 1, wherein the interface body comprises a polymeric or elastomeric material.

7. The urine collection apparatus of claim 1, wherein the cup portion and the elongate portion are overmolded over the interface body.

8. A urine collection system comprising:
a urine collection reservoir;
a urine collection apparatus for human females; and
a collection tube configured to couple to the urine collection reservoir and the urine collection apparatus and enable fluid communication therebetween, wherein
the urine collection apparatus for human females comprises:
an interface body including a cup portion and an elongate portion, the cup portion and the elongate portion defining an interface surface engaging the user's body,
wherein the cup portion is forward of the elongate portion and sized to be disposed in surrounding relation to the urethral opening of the user while the user is in a seated position, and
wherein the elongate portion is configured to be disposed under the user while the user is in a seated position.

9. The urine collection system of claim 8, wherein the collection tube comprises a plurality of bellows segments.

10. The urine collection system of claim 9, wherein the urine collection reservoir comprises a flexible containment bag.

11. The urine collection apparatus of claim 10, wherein the cup portion comprises a funnel portion, wherein the funnel portion comprises a first coupling portion at a base of the funnel portion.

12. The urine collection apparatus of claim 11, wherein the collection tube comprises a second coupling portion and a third coupling portion, wherein the plurality of bellows segments are disposed between the second coupling portion and the third coupling portion.

13. The urine collection apparatus of claim 12, wherein the urine collection reservoir comprises a fourth coupling portion, wherein each of the first coupling portion, the second coupling portion, the third coupling portion, and the fourth coupling portion comprise a threaded coupling. 14. A method of installing a urine collection system, comprising:
sliding an elongate portion of an interface body under a user while the user is in the seated position;
disposing a cup portion of the interface body in surrounding relation to a urethral opening of the user in response to sliding the elongate portion under the user;
transitioning a collection tube from a stowed condition to an expanded condition;
coupling a first coupling portion of a funnel portion of the cup portion to a second coupling portion of the collection tube; and
coupling a third coupling portion of the collection tube to a fourth coupling portion of a urine collection reservoir.
